(19) United States
(12) Patent Application Publication
(10) Pub. No.: US 2025/0186165 A1
(43) Pub. Date: Jun. 12, 2025

(54) ACTIVELY ADHERENT SURFACE FOR PARTICULATE CAPTURE

(71) Applicant: CILAG GMBH INTERNATIONAL, ZUG (CH)

(72) Inventors: VINCENT P. HARNETT, CINCINNATI, OH (US); CHRISTOPHER J. SCHALL, MASON, OH (US); BRADLEY A. ARNOLD, MASON, OH (US); LOGAN R. ROSE, LOVELAND, OH (US); DEVANATHAN RAGHAVAN, MASON, OH (US); STEVE L. SLADER, LIBERTY TOWNSHIP, OH (US); KARL W. MUELLER, CINCINNATI, OH (US); THOMAS W. LYTLE, LIBERTY TOWNSHIP, OH (US)

(21) Appl. No.: 18/530,961

(22) Filed: Dec. 6, 2023

(51) Int. Cl. A61B 90/70; A61B 17/00

(52) U.S. Cl. CPC A61B 90/70; A61B 2017/00526

(57) ABSTRACT

Surgical instruments with an actively adherent internal surface and a cover disposed within an opening is disclosed. The actively adherent internal surface and cover captures particulates that exist within the housing of the surgical instrument and limits the particles that may exit the housing from the opening.

### FIELD

The present invention generally relates to surgical instruments with adhesives applied at certain portions of the surgical instruments. More specifically, the present invention relates to actively adherent substances applied to the internal surfaces of surgical instruments so as to capture particulates.

### BACKGROUND

During the manufacturing and transportation of a surgical instrument, particulates may develop within the interior of the surgical instrument. As a result, a practitioner may observe particulates exiting the housing from an opening under the trigger or other opening within the housing of the surgical instrument. This observation can lead to concerns over the functionality of the device and may cause the practitioner to discard the device as a whole. This can lead to increased procedural times as well as a waste of materials and the discarding of a fully functional product. In some cases, having openings in the housing of a surgical instrument may be necessary for the functionality of the instrument and so it is not helpful to limit the opening unnecessarily. However, it is important to limit these particulates that may exit the housing from these openings and have them remain within the housing. The technology disclosed herein addresses the aforementioned challenges.

### SUMMARY

It is an object of the present designs to provide devices and methods to meet the above-stated needs. The designs can be for systems and devices for limiting particulates that may exit the housing of a surgical instrument through the use of actively adherent internal surfaces and a cover disposed within one or more of the openings.

The disclosed technology includes a surgical instrument. The surgical instrument comprises a hollow housing configured to receive a transmission configured to actuate an end effector. The hollow housing comprises an interior, an exterior, an opening, and an actively adherent internal surface disposed on at least some of the interior of the hollow housing. The actively adherent internal surface is configured to capture particulates that exist within the housing and to limit the particulates that may exit the housing from the opening.

The disclosed technology further includes a method for producing a surgical instrument comprising a hollow housing comprising a first shell, a second shell, an interior, an exterior, and an opening. The method includes the step of forming an actively adherent internal surface disposed on at least some of the interior of the hollow housing. The method further includes the step of connecting a selectively removable cover to a remaining portion of the opening of the housing, thereby substantially sealing the opening. The method further includes the step of connecting the second shell of the housing to the first shell of the housing.

The disclosed technology further includes a surgical instrument. The surgical instrument comprises a hollow housing configured to receive a transmission configured to actuate an end effector, the hollow housing comprising: an interior; an exterior; an opening; a trigger partially disposed within the opening of the hollow housing; a cover removably connected to the opening, the cover comprising a first side facing the interior of the hollow housing and a second side facing the exterior of the hollow housing; and an actively adherent internal surface disposed over at least some of the interior of the housing and the first side of the cover, wherein the actively adherent internal surface is configured to capture particulates that exist within the housing and to limit the particulates that may exit the housing from the opening.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is a side view illustrating the exterior of a surgical instrument, according to aspects of the present invention.

FIG. 2 is an internal view illustrating a shell of a housing of a surgical instrument, according to aspects of the present invention.

FIG. 3 is a front view a cover to be disposed within an opening of a surgical instrument, according to aspects of the present invention.

FIG. 4A is a perspective view of a surgical instrument prior to the positioning of a cover within an opening, according to aspects of the present invention.

FIG. 4B is another perspective view of the surgical instrument of FIG. 4A after a cover has been positioned within the opening, according to aspects of the present invention.

FIG. 5 is a top view illustrating the actively adherent internal surface capturing particulates, according to aspects of the present invention.

FIG. 6 is a flowchart illustrating a method of producing a surgical instrument, according to aspects of the present invention.

### DETAILED DESCRIPTION

Specific examples of the present invention are now described in detail with reference to the Figures, where identical reference numbers indicate elements which are functionally similar or identical. The examples provide solutions for surgical instrument systems that include an actively adherent internal surfaces and a cover disposed within at least one of the openings within a surgical instrument. Particulates can exist within the housing of a surgical instrument, and these particulates may exit the hollow housing from one or more openings within the housing. These particulates can be generated during the manufacturing and transportation of a surgical instrument, and a physician using the surgical instrument may notice these particulates. These particulates, although not necessarily harmful or preventative of proper use of the surgical instrument, may cause concern for the physician or other technician using the instrument, and the user may discard it.

It is therefore important that the particulates are captured within the housing of the surgical instrument so that they are prevented from exiting the housing during shipment or during use. These systems provide solutions by incorporating one or more of (a) an actively adherent internal surface disposed on an interior of the housing of the surgical instrument and/or (b) an adherent surface on the interior of a cover disposed within the opening and under the trigger. Both adherent surfaces can capture particulates that come into contact with the surface and limit an particulates that may exit the housing through the opening.

The invention is not necessarily limited to the examples described, which can be varied in construction and detail.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71.01% to 109.99%.

As used herein, the term “actively adherent” indicates a surface possessing adhesive qualities that is readily available to adhere to a material. This is not to be confused with any adhesive surface used to hold two surfaces together as the adhesive property has already been activated and is no longer ready to adhere to anything else. Further, as discussed herein, “physician” can include a doctor, surgeon, technician, scientist, operator, or any other individual or delivery instrumentation associated with delivery of a surgical instrument to a subject.

As used herein, the term “actuate” indicates the action of causing a piece of equipment to activate and achieve its intended purpose. Further, as used herein, the term “transmission” indicates a piece of equipment capable of causing an end effector to achieve its intended function by either providing power to the end effector or establishing a connection between the end effector and another piece of necessary equipment so that it can achieve its intended purpose.

As used herein, the term “particulate” indicates a loose particle of a material that exists within the housing of the surgical device. These particles serve no purpose within the surgical instrument and are only generated during the manufacturing, use or transportation of the surgical instruments. Specifically, transmissions located within the housing of the surgical instruments can generate particulates during the actuating of an end effector. Additionally, due to the moving components of the transmission possibly contacting the housing of the surgical instrument, particulates can be generated as a result. This contact can also take place as a result of vibrations produced during the transportation of the surgical instruments. These particulates can range in the size of approximately 0.5 mm by 1 mm.

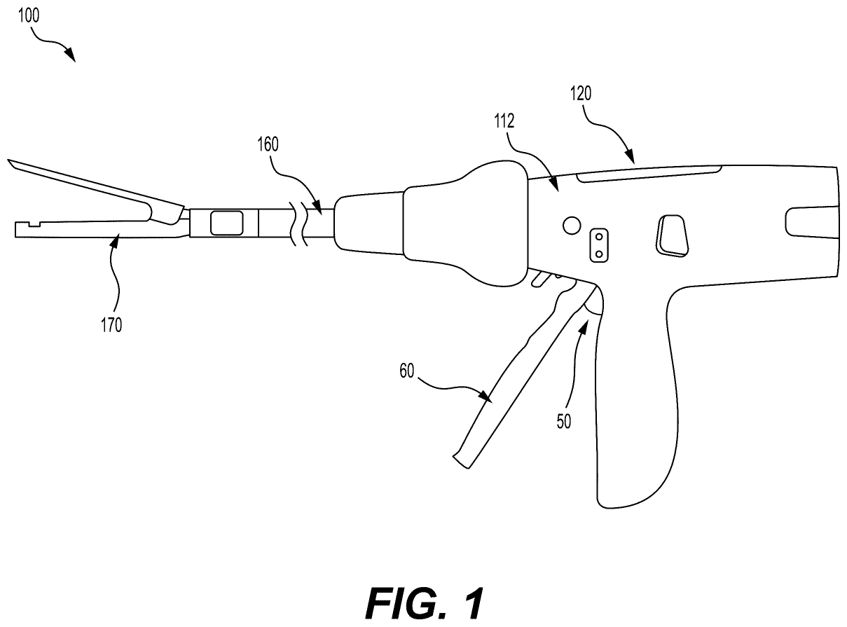

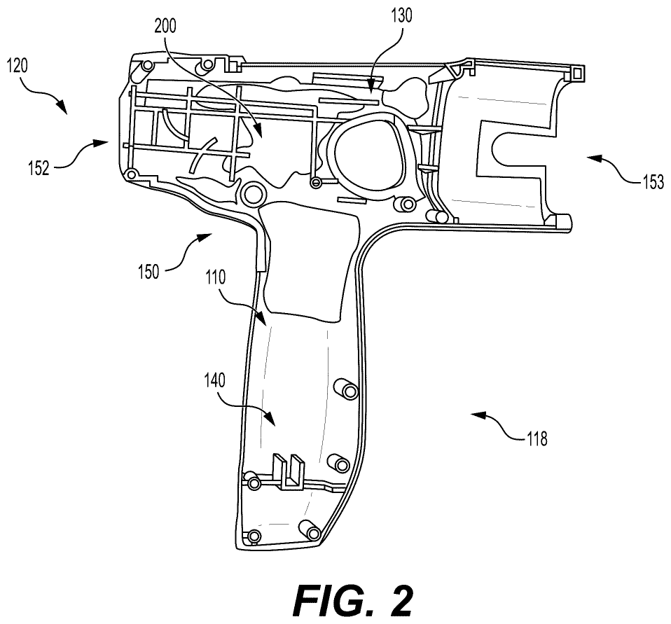

Referring now to the figures, FIG. 1 shows a side view of a surgical instrument 100. The surgical instrument 100 includes a hollow housing configured to receive a transmission configured to actuate an end effector 170. The end effector 170 can comprise multiple different devices and tools, however for the example embodiment in FIG. 1, the end effector 170 is a surgical stapler. The hollow housing 120 that forms the surgical instrument 100 is made up of two shells 116, 118, the first shell 116 and the second shell 118 being brought together to form the hollow housing 120. The hollow housing 120 comprises an interior 110 shown in further detail in FIG. 2, an exterior 112, and at least one opening 150. The hollow housing 120 further comprises an actively adherent internal surface 200 disposed over at least a portion of the interior 110 of the housing 120, the actively adherent internal surface 200 being configured to capture particulates 10 that exist within the housing 120 of the surgical instrument 100. The surgical instrument further includes a cover 50 disposed at least partially within the opening 150 of the hollow housing 120 and a trigger 60 also disposed within the opening 150 such that the cover 50 interferes with the movement of the trigger 60 when disposed in the opening 150.

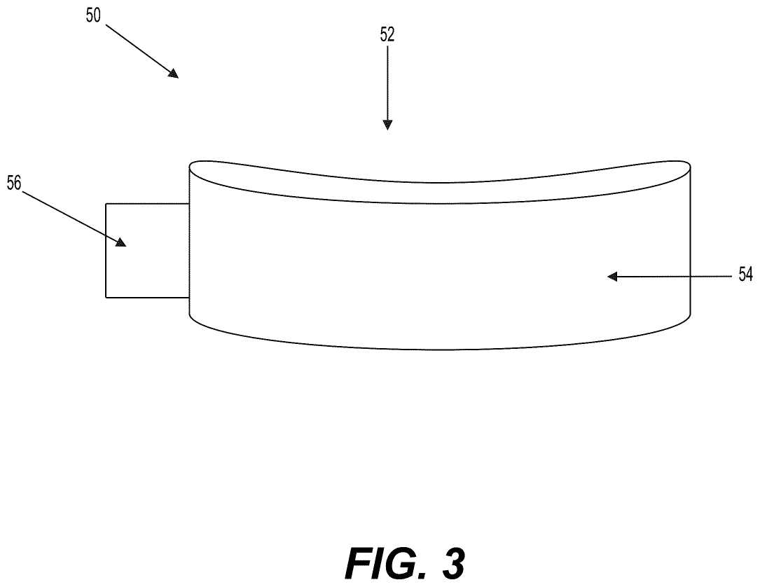

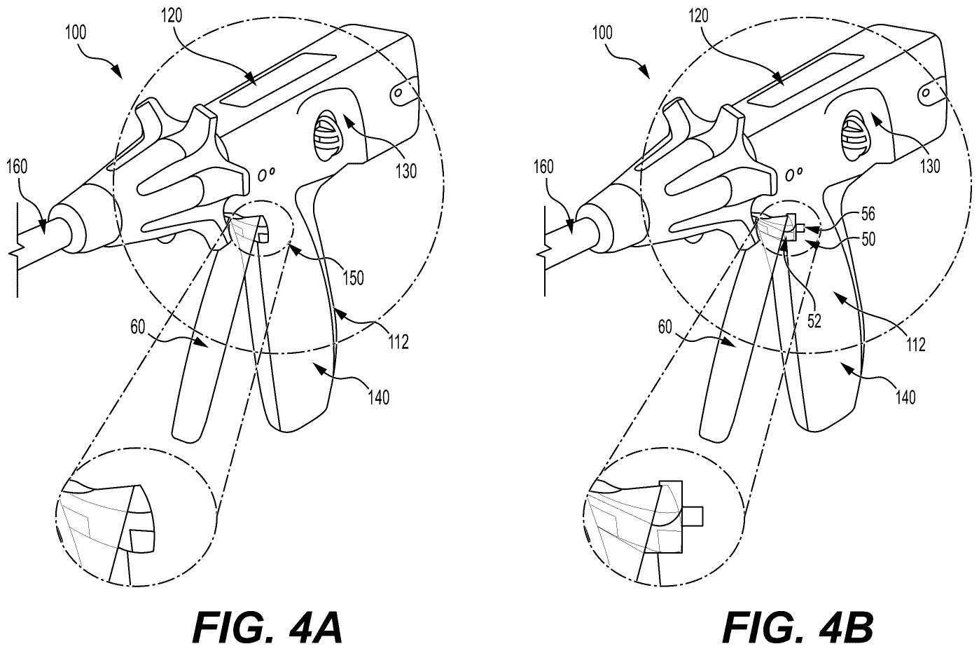

The actively adherent internal surface 200 captures the particulates 10 that come in contact with the surface 200. The actively adherent internal surface 200 of the surgical instrument 100 should be able to withstand a sterilization process in the manufacturing of a surgical instrument 100 as well as be able to withstand extreme temperature variations. Example sterilization processes that the adherent internal surface 200 should withstand include Gamma or Ethylene Oxide sterilization. Further, the actively adherent internal surface 200 is configured to capture particulates 10 having dimensional parameters of approximately 0.5 mm by 1 mm. Particulates 10 that are generated during the manufacturing and transportation of the surgical instrument 100 are loose within the housing 120 and as the surgical instrument 100 is moved around, and the particulates 10 move around the housing 120 and contact the interior 110 walls of the housing 120. Having an actively adherent internal surface 200 disposed over the interior 110 of the housing 120 causes the particulates 10 to stick to the surfaces 200 that they contact. These particulates 10 are then prevented from exiting the housing 120 of the surgical instrument 100 at any time. As will be explained further in FIGS. 3 and 4A-4B, the cover 50 disposed within the opening 150 of the housing 120 also limits the quantity of particulates that may exit the housing 120 through that opening 150 with an actively adherent internal surface 200 disposed on a side 52 of the cover 50.

Turning our attention again to FIG. 2, the figure shows the interior 110 of the second shell 118 of the surgical instrument 100 depicted in FIG. 1. The hollow housing 120 further includes an actively adherent internal surface 200 disposed over at least a portion of the interior 110 of the hollow housing 120. The hollow housing 120 can further comprise a shaft portion 130 having an external surface 134 and an internal surface 132 and a handle portion 140 having an external surface 144 and an internal surface 142. The actively adherent internal surface 200 can be disposed over the interior 110 of the hollow housing 120 such that the actively adherent internal surface 200 is disposed over at least some of the internal surface 132 of the shaft portion 130 and at least some of the internal surface 142 of the handle portion 140 of the hollow housing 120. The actively adherent internal surface 200 is configured to capture particulates 10 that exist within the hollow housing 120 and limit any particulates 10 that may exit through an opening 150 of the surgical instrument 100. The actively adherent internal surface 200 disposed on each internal surface 132, 142 of the shaft 130 and handle portion 140 portion respectively, may also be contiguous to from a constant actively adherent internal surface 200 across the interior 110 of the hollow housing 120 including shell 116.

As shown in FIGS. 1, 2, 4A, 4B and 5, the hollow housing 120 of the surgical instrument 100 includes at least one opening 150, the opening 150 located within the handle portion 140 of the hollow housing 120. A trigger 60 and a cover 50 are disposed within the first opening 150. The hollow housing 120 can further include a second opening 152 located within the shaft portion 130 of the hollow housing 120. In some examples, a shaft 160 having an end effector 170 can be attached to the second opening 152 of the hollow housing 120. Additionally, the hollow housing 120 can further include a third opening 153 located within the shaft portion 130 of the hollow housing 120. In some examples, a battery pack (not illustrated) can be inserted into the third opening 153 of the hollow housing 120.

FIG. 3 illustrates a cover 50 that can be removably connected to the opening 150 of a hollow housing 120, wherein a trigger 60 can also be positioned within the opening 150. The cover 50 is disposed within the opening 150 such that the movement of the trigger 60 is limited by the cover 50 until the cover 50 is removed. This is due to the fact that, prior to use of the surgical instrument 100, the cover 50 is removed from the opening 150 of the hollow housing 120 to give the trigger 60 free range of movement again. FIGS. 4A and 4B provide additional illustrations of attachment and removal of the cover 50. The cover 50 comprises a first side 52 facing towards the interior 110 of the hollow housing 120 and a second side 54 facing away from the surgical instrument 100. The cover also comprises a tab 56 disposed on the second side of the cover 50.

The cover 50 also includes an actively adherent internal surface 200 disposed over the first side 52 of the cover. The actively adherent internal surface 200 of the cover 50 serves the purpose of limiting any particulates 10 that may exit the hollow housing 120 through the first opening 150 by capturing particulates 10 that come in contact with the actively adherent internal surface 200 disposed on the first side 52. The cover 50 is to be removed along with the captured particulates 10 prior to operation of the surgical instrument 100.

FIGS. 4A-4B illustrate the attachment of the cover 50 within the opening 150 of the hollow housing 120. In FIG. 4A, the hollow housing 120, as described above, comprises a trigger 60 disposed within the opening 150, the trigger 60 having full range of movement when cover 50 is not present to restrict such movement. In FIG. 4B, the cover 50 is removably connected to the opening 150 in the hollow housing 120, thereby interfering with the movement of the trigger 60. This is to prevent the trigger 60 from moving out of place and to allow the cover 50 to fully occupy the remaining space so that the actively adherent internal surface 200 disposed on the first side 52 of the cover can capture particulates 10 that contact the first side 52. This will limit particulates 10 that may have exited the hollow housing 120 through the opening 150. A tab 56 is disposed on the second side 54 of the cover 50 and someone operating the surgical instrument 100 will pull on this tab 56 in order to remove the cover 50 from the opening 150 prior to the use of the surgical instrument 100.

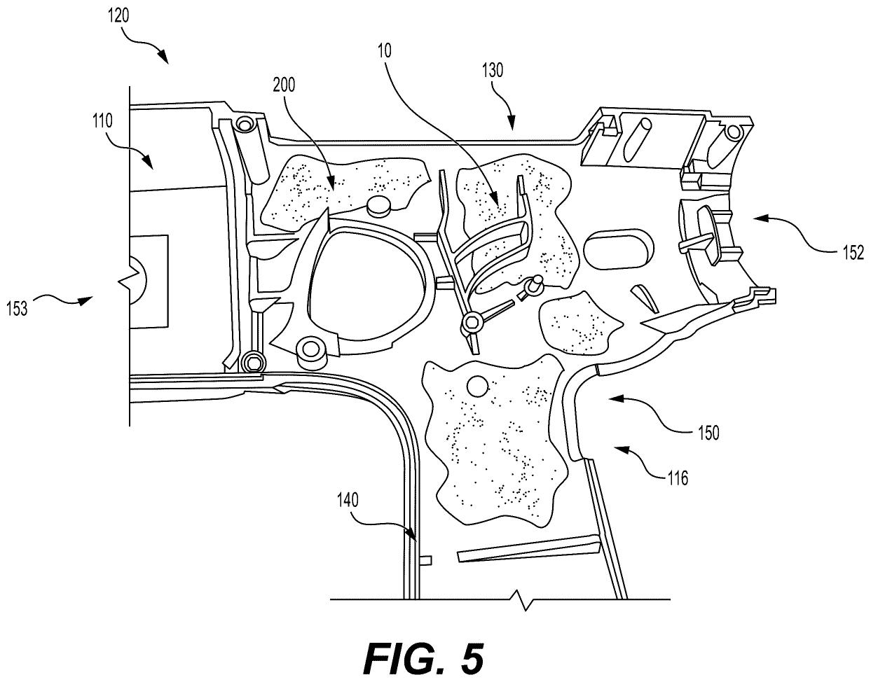

FIG. 5 illustrates an interior 110 of a first shell 116 of a hollow housing 120 for a surgical instrument 100 that is to be joined to the second shell 118 illustrated in FIG. 2. The hollow housing 120 can be categorized by a shaft portion 130 defining the upper half of the shell 116 and a handle portion 140 defining the bottom half of the shell 116. The shaft portion 130 comprises an internal surface 132 and an external surface 134 and the handle portion 140 comprises an internal surface 142 and an external surface 144. The hollow housing 120 further comprises an actively adherent internal surface 200 disposed over at least a portion of the internal surface 132 of the shaft portion 130 and the internal surface 142 of the handle portion 140.

The actively adherent internal surface 200 can be contiguous across the interior 110 of the hollow housing 120 to from a constant adherent surface across the interior 110. This allows for the actively adherent internal surface 200 to cover a wide enough area and increase the number of particulates 10 that can be captured by coming into contact with the actively adherent internal surface 200. The actively adherent internal surface 200 can be disposed over additional areas of the interior 110 of the hollow housing 120 that do not include the shaft portion 130 or the handle portion 140. For example, the interior of a shaft 160 may have an actively adherent internal surface 200 disposed within it to prevent particulates that may exist within the shaft 160 from exiting the shaft 160.

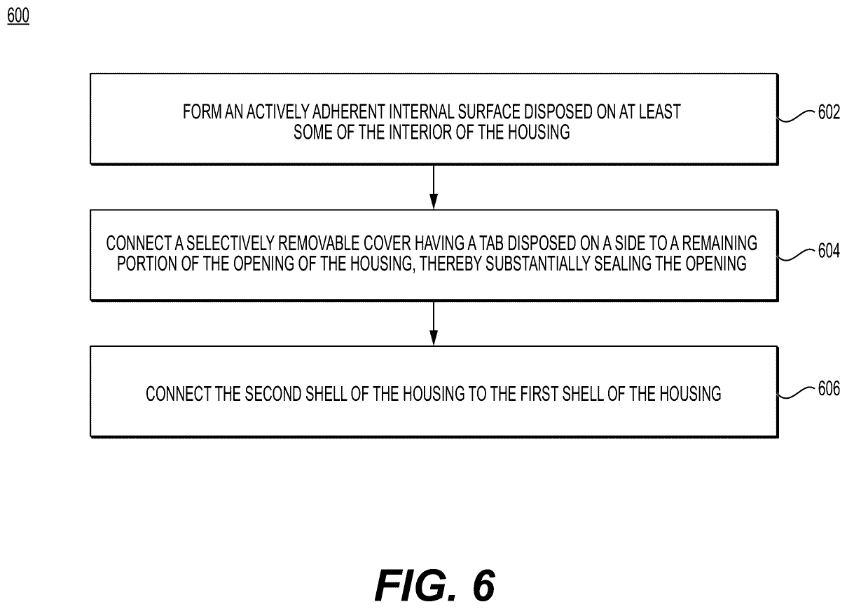

FIG. 6 shows an example method 600 for producing a surgical instrument 100. The method 600 can be performed on any of the surgical instruments described herein, including for example a surgical instrument 100 comprising a hollow housing 120 having a first shell 116 and a second shell 118, each shell 116, 118 comprising a handle portion 140, a shaft portion 130, an interior 110, an exterior 112, and an opening 150.

The method 600 includes the step of forming 602 an actively adherent surface 200 disposed on at least some of the interior 110 of the housing 120. The method 600 further includes the step of connecting 604 a selectively removable cover 50 having a tab 56 disposed on a side to a remaining portion of the opening of the housing 120, thereby substantially sealing the opening 150. The method 600 further includes the step of connecting 606 the second shell 118 of the housing 120 to the first shell 116 of the housing 120.

The method 600 further includes the step of capturing particulates 10 on the actively adherent internal surface 200 that exist within the housing 120. The method 600 further includes the step of preventing particulates 10 from exiting the housing 120 from the opening 150 with the cover 50 disposed in the opening 150 and removing the cover 50 by pulling the tab 56 disposed on the side of the cover 50.

Examples of the present disclosure can be implemented by any of the following numbered clauses:

Clause 1: A surgical instrument (100), comprising: a hollow housing (120) configured to receive a transmission configured to actuate an end effector (170), the hollow housing (120) comprising: an interior (110); an exterior (112); an opening (150); and an actively adherent internal surface (200) disposed on at least some of the interior (110) of the hollow housing (120), wherein the actively adherent internal surface (200) is configured to capture particulates (10) that exist within the housing (120) and to limit the particulates (10) that may exit the housing (120) from the opening (150).

Clause 2: The surgical instrument (100) of clause 1, further comprising a cover (50) removably connected to the opening (150) of the housing (120) thereby substantially sealing the opening (150), the cover (50) capable of limiting particulates (10) that may exit the housing (120) via the opening (150).

Clause 3: The surgical instrument (100) of clause 2, wherein the cover (50) comprises a first side (52) facing the interior (110) of the housing (120) of the surgical instrument (100) and a second side (54) facing the exterior (112) of the housing (120) of the surgical instrument (100).

Clause 4: The surgical instrument (100) of any one of clauses 2-3, wherein the actively adherent internal surface (200) is further disposed on the first side (52) of the cover (50) and configured to limit particulates (10) exiting the housing (120) from the opening (150) by capturing particulates (10) that come in contact with the actively adherent internal surface (200).

Clause 5: The surgical instrument (100) of any one of clauses 2-4, further comprising a tab (56) disposed on the second side (54) of the cover (50) and configured to remove the cover (50) prior to using the surgical instrument (100).

Clause 6: The surgical instrument (100) of any one of clauses 2-5, further comprising a trigger (60) disposed within the opening (150); wherein the cover (50) interferes with the movement of the trigger (60) when disposed in the opening (150).

Clause 7: The surgical instrument (100) of any one of the preceding clauses, wherein the actively adherent internal surface (200) is configured to capture particulates (10) having dimensional parameters of at least 0.5 mm by 1 mm.

Clause 8: The surgical instrument (100) of any one of the preceding clauses, wherein the actively adherent internal surface (200) is configured to withstand a sterilization process in the manufacture of the surgical instrument (100) and extreme temperature variation.

Clause 9: The surgical instrument (100) of any one of the preceding clauses, wherein the actively internal surface (200) is contiguous.

Clause 10: The surgical instrument (100) of clause 1, wherein the end effector (170) is a surgical stapler.

Clause 11: The surgical instrument (100) of clause 1, wherein the hollow housing (120) further comprises a handle portion (140) having an internal surface (142) and an external surface (144) and a shaft portion (130) having an internal surface (132) and an external surface (134).

Clause 12: The surgical instrument (100) of clause 11, wherein the shaft portion (130) of the housing (120) can comprise a second opening (152) and a third opening (153).

Clause 13: The surgical instrument (100) of any one of clauses 11-12, wherein the actively adherent internal surface (200) is disposed on at least some of the internal surface (142) of the handle portion (140) and at least some of the internal surface (132) of the shaft portion (130).

Clause 14: The surgical instrument (100) of any one of clauses 11-13, wherein the actively adherent surface (200) disposed on the internal surface (142) of the handle portion (140) and the internal surface (132) of the shaft portion (130) is contiguous.

Clause 15: A method (600) for assembling a surgical instrument (100), the surgical instrument (100) comprising: a hollow housing (120) comprising a first shell (116), a second shell (118), an interior (110), an exterior (112), and an opening (150), the method (600) comprising the steps of: forming an actively adherent internal surface (200) disposed on at least some of the interior (110) of the hollow housing (120) (502); connecting a selectively removable cover (50) to a remaining portion of the opening (150) of the housing (120), thereby substantially sealing the opening (150) (504); and connecting the second shell (118) of the housing (120) to the first shell (116) of the housing (120) (506).

Clause 16: The method (600) of clause 15, further comprising the step of: capturing particulates (10) on the actively adherent internal surface (200) that exist within the housing (120) of the surgical instrument (100).

Clause 17: The method (600) of any one of clauses 15-16, wherein the actively adherent internal surface (200) is configured to withstand a sterilization process in the manufacture of the surgical instrument (100) and extreme temperature variation.

Clause 18: The method (600) of any one of clauses 15-17, further comprising the step of: preventing particulates (10) from exiting the housing (120) via the opening (150), when the cover (50) is disposed in the opening (150).

Clause 19: The method (600) of any one of clauses 15-18, wherein the cover (50) comprises a first side (52) facing the interior (110) of the housing (120) of the surgical instrument (100) and a second side (54) facing the exterior (112) of the housing (120) of the surgical instrument (100).

Clause 20: The method (600) of clause 19, further comprising the step of: forming an actively adherent internal surface (200) on the first side (52) of the cover (50) to capture particulates (10) that exist within the housing (120) of the surgical instrument (100).

Clause 21: The method (600) of clause 20, disposing a tab (56) on the second side (54) of the cover (50) that is configured to remove the cover (50) and removing the cover (50) by pulling the tab (56) to expose the remaining portion of the opening (150) prior to using the surgical instrument (100).

Clause 22: The method (600) of clause 15, wherein the housing further comprises a second opening (152) and a third opening (153); further comprising the step of: connecting a shaft (160) to the housing (120) at the second opening (152); and connecting a trigger (60) to the housing (120) at the first opening (150).

Clause: 23: The method (600) of any one of clauses 15-22, wherein the end effector (170) is a surgical stapler.

Clause 24: A surgical instrument (100), the surgical instrument (100) comprising: a hollow housing (120) configured to receive a transmission configured to actuate an end effector (170), the hollow housing (120) comprising: an interior (110); an exterior (112); an opening (150); a trigger (60) partially disposed within the opening (150) of the hollow housing (120); a cover (50) removably connected to the opening (150), the cover (50) comprising a first side (52) facing the interior (110) of the hollow housing (120) and a second side (54) facing the exterior (112) of the hollow housing (120); and an actively adherent internal surface disposed over at least some of the first side (52) of the cover (50), wherein the actively adherent internal surface (200) is configured to capture particulates (10) that exist within the housing (120) and to limit the particulates (10) that may exit the housing (120) from the opening (150).

Clause 25: The surgical instrument (100) of clause 24, wherein the cover (50) comprises a tab (56) disposed on the second side (54) of the cover (50) and configured to remove the cover (50) from the opening (150) prior to using the surgical instrument (100).

Clause 26: The surgical instrument (100) of any one of clauses 24-25, wherein the actively adherent internal surface (200) is further disposed on at least some of the interior (110) of the hollow housing (120).

Clause 27: The surgical instrument (100) of clause 24, wherein the actively adherent internal surface (200) disposed on the at least some of the interior (110) of the hollow housing (120) is contiguous across the interior (110).

Clause 28: The surgical instrument (100) of any one of clauses 24-27, wherein the actively adherent internal surface (200) is configured to capture particulates (10) having dimensional parameters of at least 0.5 mm by 1 mm.

Clause 29: The surgical instrument (100) of any one of clauses 24-28, wherein the actively adherent internal surface (200) is configured to withstand a sterilization process in the manufacture of the surgical instrument (100) and extreme temperature variation.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

1-14. (canceled)

15. A surgical instrument, comprising:
a hollow housing configured to receive a transmission configured to actuate an end effector, the hollow housing comprising:
an interior;
an exterior;
an opening; and
an actively adherent internal surface disposed on at least some of the interior of the hollow housing, wherein the actively adherent internal surface is configured to capture particulates that exist within the housing and to limit the particulates that may exit the housing from the opening.

16. The surgical instrument of claim 15, further comprising a cover removably connected to the opening of the housing thereby substantially sealing the opening, the cover capable of limiting particulates that may exit the housing via the opening.

17. The surgical instrument of claim 16, wherein the cover comprises a first side facing the interior of the housing of the surgical instrument and a second side facing the exterior of the housing of the surgical instrument.

18. The surgical instrument of claim 16, wherein the actively adherent internal surface is further disposed on the first side of the cover and configured to limit particulates exiting the housing from the opening by capturing particulates that come in contact with the actively adherent internal surface.

19. The surgical instrument of claim 16, further comprising a tab disposed on the second side of the cover and configured to remove the cover prior to using the surgical instrument.

20. The surgical instrument of claim 19, further comprising a trigger disposed within the opening;
wherein the cover interferes with the movement of the trigger when disposed in the opening.

21. The surgical instrument of claim 15, wherein the actively adherent internal surface is configured to capture particulates having dimensional parameters of at least 0.5 mm by 1 mm.

22. The surgical instrument of claim 15, wherein the actively adherent internal surface is configured to withstand a sterilization process in the manufacture of the surgical instrument and extreme temperature variation.

23. The surgical instrument of claim 15, wherein the hollow housing further comprises a handle portion having an internal surface and an external surface and a shaft portion having an internal surface and an external surface.

24. The surgical instrument of claim 23, wherein the shaft portion of the housing can comprise a second opening and a third opening.

25. The surgical instrument of claim 23, wherein the actively adherent internal surface is disposed on at least some of the internal surface of the handle portion and at least some of the internal surface of the shaft portion.

26. The surgical instrument of claim 25, wherein the actively adherent surface disposed on the internal surface of the handle portion and the internal surface of the shaft portion is contiguous.

27. A method for assembling a surgical instrument, the surgical instrument comprising:
a hollow housing comprising a first shell, a second shell, an interior, an exterior, and an opening,
the method comprising the steps of:
forming an actively adherent internal surface disposed on at least some of the interior of the hollow housing;
connecting a selectively removable cover to a remaining portion of the opening of the housing, thereby substantially sealing the opening; and
connecting the second shell of the housing to the first shell of the housing.

28. The method of claim 27, further comprising the step of: capturing particulates on the actively adherent internal surface that exist within the housing of the surgical instrument.

29. The method of claim 27, wherein the actively adherent internal surface is configured to withstand a sterilization process in the manufacture of the surgical instrument and extreme temperature variation.

30. The method of claim 27, further comprising the step of: preventing particulates from exiting the housing via the opening, when the cover is disposed in the opening.

31. The method of claim 30, wherein the cover comprises a first side facing the interior of the housing of the surgical instrument and a second side facing the exterior of the housing of the surgical instrument.

32. The method of claim 31, further comprising the step of: forming an actively adherent internal surface on the first side of the cover to capture particulates that exist within the housing of the surgical instrument.

33. The method of claim 32, disposing a tab on the second side of the cover that is configured to remove the cover and removing the cover by pulling the tab to expose the remaining portion of the opening prior to using the surgical instrument.

34. The method of claim 27, wherein the housing further comprises a second opening and a third opening; further comprising the step of: connecting a shaft to the housing at the second opening; and connecting a trigger to the housing at the first opening.
